(19) United States
(12) Patent Application Publication
(10) Pub. No.: US 2009/0105602 A1
(43) Pub. Date: Apr. 23, 2009

(54) Staged life-threatening arrhythmia detection algorithm for minimizing power consumption

(75) Inventors: Stacy Gehman, Seattle, WA (US); Thomas Lyster, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS, N.V., EINDHOVEN (NL)

(21) Appl. No.: 10/598,323

(22) PCT Filed: Feb. 11, 2005
(86) PCT No.: PCT/IB2005/050537

§ 371 Date: Aug. 24, 2006

Related U.S. Application Data
(60) Provisional application No. 60/548,850, filed on Feb. 27, 2004.

(51) Int. Cl. A61B 5/0464; A61B 5/046

(52) U.S. Cl. 600/516; 600/508; 600/518

(57) ABSTRACT

A two-stage digital algorithm uses a highly sensitive low power digital first stage to detect one or more alarm conditions, and one or more complex digital subsequent stages that identify the detected alarm condition with more specificity. The one or more complex digital subsequent stages are not activated, and consume no power, until an alarm condition is sensed by the low power consumption digital first stage. Given that the second stage will process the data more rigorously, the low power first stage can be set to be more sensitive and generate what would otherwise be excessive alarms, which are ultimately filtered out by the subsequent stages. By staging the digital analysis algorithms, the present invention achieves high sensitivity for alarm conditions with low computational throughput and low power consumption, and achieves high specificity with more computationally intensive algorithms that only run occasionally.

The present invention relates generally to methods and apparatus for monitoring physiological conditions, and more particularly to a method and apparatus for monitoring a physiological condition, such as a cardiac condition, of a wearer of a portable device.

Monitoring the physiological state of an individual enables rapid detection of potentially life threatening events, particularly those that can be predicted from certain trends. To enable more continuous monitoring, devices have been developed that can be worn.

However, monitoring or alarm devices that are to be worn on one's body must overcome certain design challenges. In general, a body worn device must be small and lightweight so that one can wear the device in comfort. Moreover, a body worn device must be highly sensitive at detecting alarm conditions to avoid missing alarms, yet a body worn device must also be highly specific at detecting alarm conditions to avoid excessive false alarms.

The concomitant demand for high sensitivity and high specificity typically leads to algorithms that require high computational throughput. Unfortunately, high computational throughput in digital devices generally requires high power consumption, which in turn leads to larger, heavier power sources to support this high computational throughput. Thus, the demand for high sensitivity and high specificity has generally precluded development of a small, lightweight, yet comfortable body worn device.

Yet the need remains for a small, portable monitoring device that is both highly sensitive and highly specific.

The present invention is therefore directed to the problem of developing a method and apparatus for increasing the sensitivity and specificity of a small, portable, body worn monitoring device without also increasing the power consumption.

The present invention addresses these and other problems by providing a multi-stage digital algorithm with a highly sensitive low power digital first stage to detect one or more alarm conditions, and one or more complex digital subsequent stages that identify the detected alarm condition with more specificity. According to one aspect of the present invention, the complex digital subsequent stages are not activated, and therefore consume no power, until an alarm condition is sensed by the low power consumption digital front end. Given that the subsequent stages will process the data more rigorously, the low power first stage can be set to be more sensitive and generate what would otherwise be unwarranted alarms, which are ultimately filtered out by the subsequent stage.

The present invention, by staging the digital analysis algorithms, achieves high sensitivity for alarm conditions with low computational throughput and low power consumption, and achieves high specificity with more computationally intensive algorithms that only run occasionally, thus achieving minimum power consumption with both high sensitivity and high specificity.

These and other advantages will be apparent upon review of the detailed description in light of the following drawings, in which:

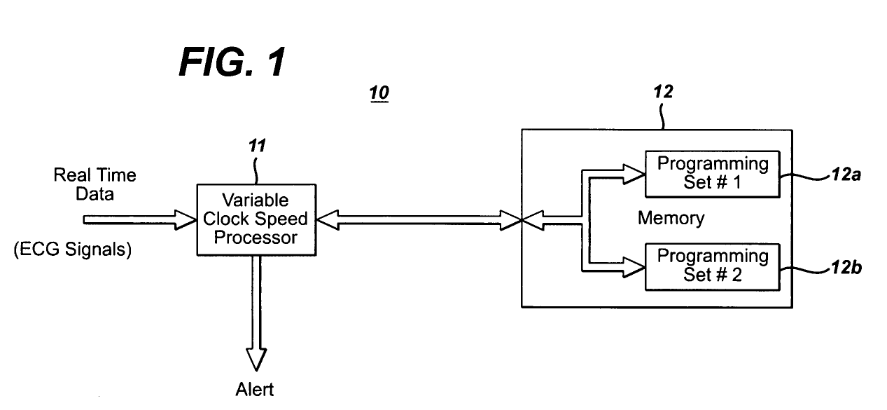

FIG. 1 illustrates an exemplary embodiment of an apparatus for monitoring a physiological condition of a wearer according to one aspect of the present invention.

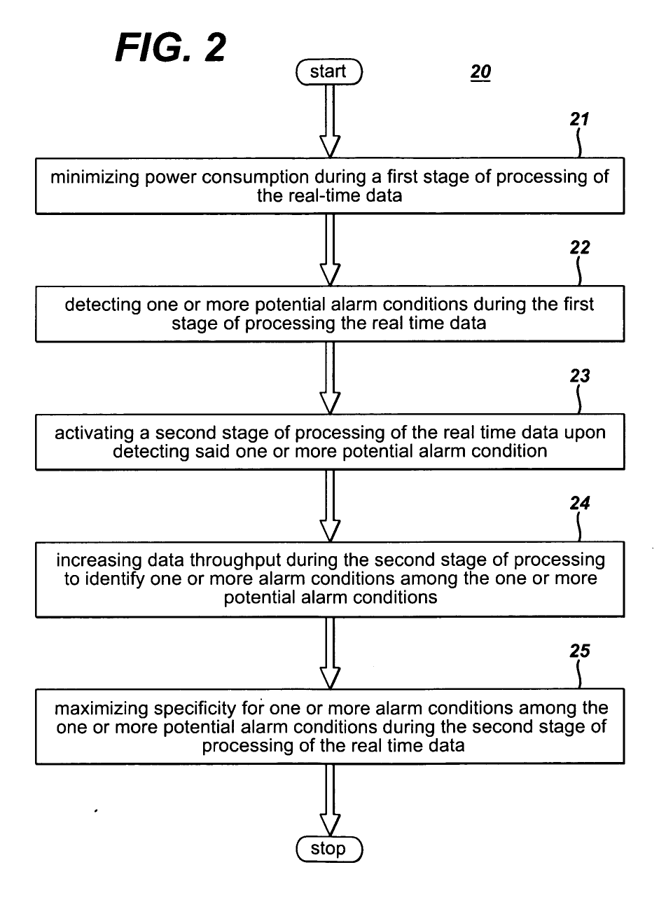

FIG. 2 illustrates an exemplary embodiment of a method for monitoring a physiological condition of a wearer according to another aspect of the present invention.

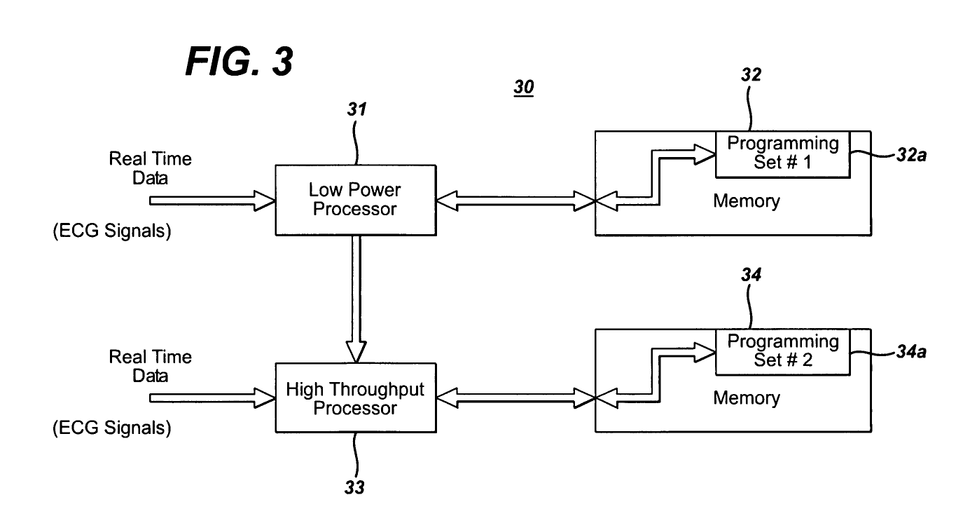

FIG. 3 illustrates an exemplary embodiment of an apparatus for monitoring a physiological condition of a wearer according to yet another aspect of the present invention.

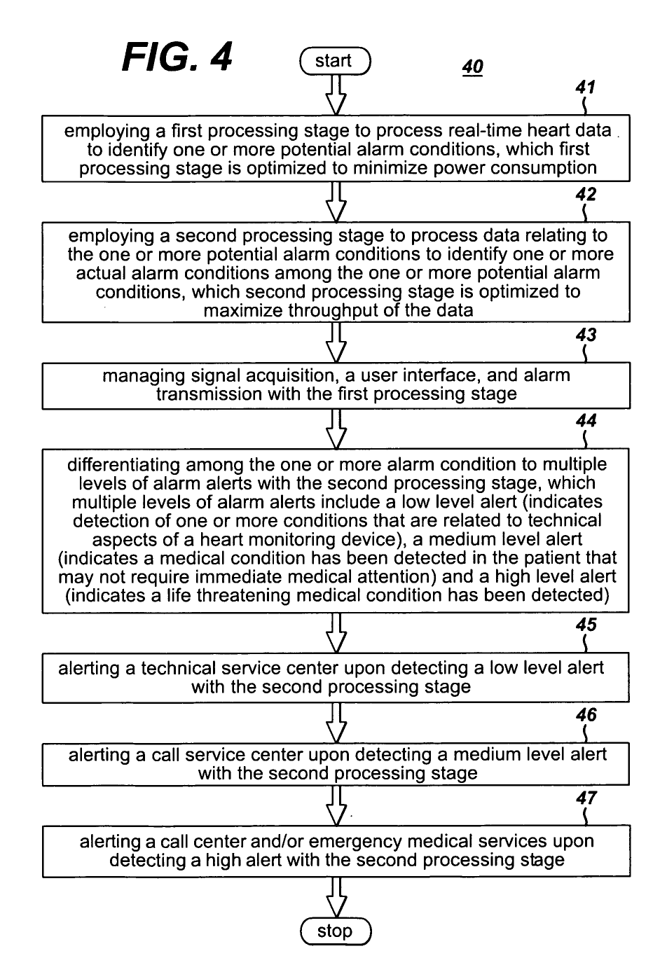

FIG. 4 illustrates an exemplary embodiment of a method for monitoring a physiological condition of a wearer according to still another aspect of the present invention.

One technique for reducing the power consumption of body worn monitoring devices is to employ an analog electrocardiogram (ECG) QRS detector that is used to determine if the heart rate is within normal bounds. The analog detector triggers a digital analysis algorithm when the heart rate is not within normal bounds. However, the embodiments of the present invention have the advantage that more complex algorithms can be practically implemented digitally than by using analog devices, so that sensitivity and specificity for the alarm conditions of the first stage algorithm can be greater than that of an analog QRS detector, thus maximizing the time spent in low power mode.

One aspect of the present invention includes an algorithm that is designed to detect one or more alarm conditions. This algorithm employs multiple stages for processing data in real time, and minimizes power consumption by, for example, varying the processor clock speed for the algorithm stages. For example, in a first detection stage, the processor clock speed (or the processor itself) is selected to maintain the power consumption of the processor at a very low level. In contrast, in one or more subsequent stages that are activated upon detection of some event detected by the first stage, the processor clock speed (or the processor itself) is then increased to optimize the computational power of the subsequent stage to enable complex or high-powered algorithms to be levied against the incoming data to identify with high accuracy certain specific conditions or events. In another example, because of the low computational throughput needed by the first stage, power consumption in the first stage can be minimized without altering the processor clock speed, by putting the processor in very low power “standby mode” when the first stage computations have been completed. In this example power consumption during each stage is determined by how long it takes to execute the stage, with the first stage requiring much less time to execute than the subsequent stages.

According to another aspect of the present invention, the first stage optimizes sensitivity for the alarm conditions and minimizes computational throughput, and hence minimizes power consumption. Within these constraints, the first stage achieves the highest possible sensitivity for the alarm conditions to maximize the time spent in the first stage while minimizing the amount of power consumed in this stage. By maximizing the amount of time spent in the first stage, which draws minimal power, relative to the subsequent stages, which draw significantly more power, the portable device can be designed with a power source that is small, compact and remains consistent with a body worn portable device.

According to another aspect of the present invention, subsequent stages optimize specificity for the alarm conditions, with algorithm throughput constrained only by maximum processor clock speed and power budget. As these subsequent stages are not powered except in certain infrequent instances, the power consumption of these subsequent stages is not significant relative to the total power consumption of the device.

Turning to FIG. 1, shown therein is a block diagram of an exemplary embodiment 10 of a processing portion of a wearable physiological sensor. According to this embodiment 10, the first stage of the algorithm (stored, e.g., in memory 12 in programming instructions set #1, 12a) will detect life-threatening arrhythmias. The life-threatening arrhythmia detection (LTAD) algorithm 12a will run on a microprocessor 11 which can be controlled automatically so that the microprocessor 11 can be set to a minimal power consumption mode by either of the two exemplary methods, i.e., by controlling the clock speed or by entering standby mode, or by a combination of the two methods. An example of such a microprocessor includes a microprocessor manufactured by Texas Instruments, Model No. MSP430F149. In its active mode, the power consumption of this processor depends on its clock speed (typically around 1.5 MHz); this microprocessor also has a very low power standby mode (typically less than 2 microamperes), with an internal timer that terminates standby mode at programmable intervals.

In addition to the LTAD algorithm 12a, the microprocessor 11 will manage other tasks, including signal acquisition and processing (e.g., ECG and artifact reference signals), a user interface, and alarm transmission. The programming instructions for these tasks can be embedded in programming instruction set #1. Stage one of the LTAD algorithm 12a plus these other tasks will determine the minimum computational throughput of the processor 11, which will also determine the minimum clock rate needed to maintain real-time operation or the time the processor spends in active mode while the computations are completed, followed by standby mode.

Although multiple stages for the LTAD algorithm are possible, two stages (12a, 12b) are probably adequate in most cases. In this embodiment 10, the first stage 12a is optimized for sensitivity for alarm detection and low computational throughput, while the second stage algorithm 12b (stored in programming instruction set #2) is optimized for specificity of alarm detection and executes at a higher clock rate. For example, as part of programming instructions set #2, the execution of which is activated by detection of one or more potential alarm conditions during execution of programming instructions set #1, the processor will increase its own clock rate to a value that will maximize the data throughput of the processor. Alternatively, if a fixed clock rate is used by all stages, the stages subsequent to the first will execute longer (relative to the first stage) in active mode before entering standby mode. For a typical balance of the first stage relative to subsequent stages, with subsequent stages running 1% of the time or less, the exemplary processor (e.g., TI MSP430F149) will average less than 50 microamperes in current drain.

Stage one of the LTAD algorithm 12a can detect life-threatening arrhythmias, such as ventricular fibrillation (VF), fast ventricular tachycardia (VT), extreme bradycardia and asystole. High sensitivity will be achieved in the first stage algorithm 12a. According to another aspect of the present invention, the first stage algorithm 12a uses ECG data as its primary input. These alarm conditions can be sensitively detected with a QRS detector/counter for estimating heart rate, and with rate thresholds for the various conditions. QRS detectors are described in the text “Biomedical Digital Signal Processing: C Language Examples and Laboratory Experiments for the IBM PC,” Willis J. Tompkins, ed. (Prentice Hall, 1993). Rate-based algorithms alone, however, are prone to false detection of ventricular fibrillation and fast ventricular tachycardia due to contamination of the ECG signal by motion artifact during exertion by the patient.

According to another aspect of the present invention, the second stage 12b of the LTAD algorithm uses: (1) independent estimates of the rate to confirm that thresholds have been exceeded; (2) other parameters estimated from the ECG related to VF and VT; and (3) a signal derived from common mode currents (CMC); in the signal acquisition module to indicate patient motion or disturbance (which is described in U.S. Pat. No. 5,902,249 entitled “Method and Apparatus for Detecting Artifacts Using Common-Mode Signals in Differential Signal Detectors,” which is hereby incorporated by reference as if repeated herein in its entirety, including the drawings).

Other signals, such as acceleration or patient impedance may also be used to indicate artifacts (which are described in U.S. Pat. No. 6,287,328 entitled “Multiple Artifact Assessment,” which is hereby incorporated by reference as if repeated herein in its entirety, including the drawings).

Turning to FIG. 2, shown therein is an exemplary embodiment of a method 20 for monitoring real time data signals from a heart in a body worn, portable device. This method can be employed by the embodiment of FIG. 1 or any apparatus set forth herein.

In step 21, power consumption is minimized during a first stage of processing of the real-time data. This can be accomplished either by selection of the processor or by controlling a processor to operate in a low power mode.

In step 22, one or more potential alarm conditions are detected during the first stage of processing the real time data. The one or more potential alarm conditions that can be detected are discussed later.

In step 23, a second stage of processing of the real time data is activated upon detecting said one or more potential alarm conditions. The activation can consist of activating a second processor, activating a second algorithm, increasing a clock speed, or downloading a second programming set of instructions, as well as other techniques.

In step 24, data throughput is increased during the second stage of processing to identify one or more alarm conditions among the one or more potential alarm conditions. Data throughput is increased to enable complex algorithms to be employed against the incoming data as discussed subsequently.

In step 25, specificity for one or more alarm conditions among the one or more potential alarm conditions is maximized during the second stage of processing of the real time data. The process of identifying the actual alarm conditions is set forth more precisely below. Detection of artifacts present in the data can be used to filter out extraneous alarms, but this requires the higher computational throughput of the second stage processing.

Alternatively, rather than varying the clock speed of one processor, multiple processors could be used for the algorithm stages, with the throughput (and power consumption) matched to the needs of the algorithm stages. Turning to FIG. 3, shown therein is an exemplary embodiment 30 of a processing section of a body worn, portable heart-monitoring device. In this embodiment 30, a low power, low voltage (and likely low computational throughput) processor 31 is used for the first stage algorithm 32a (which is stored, for example, in memory 32) to process the incoming data in real time. An example of a low power processor includes the microprocessor described earlier by Texas Instruments.

Upon detection of some event requiring further analysis, the first processor 31 activates a second processor 33. Processor 33 is a high computational throughput processor that is selected for the second stage algorithm 34a (which is stored, for example, in memory 34) to enable high-powered algorithms to be employed against the incoming data to ensure accurate identification of any events in the incoming data. An example of a high computational processor includes the same Texas Instruments microprocessor being clocked at a higher clock rate.

While the embodiment 30 shows two memories 32, 34 a single memory could be employed that is accessible by each of the processors 31, 33 as necessary to access algorithms 32a and 34a.

While the incoming data in FIG. 3 is shown being applied in parallel to both processors 31, 33 (in which case, high computational processor 33 does not operate until activated by low power processor 31), the incoming data could be passed from the low power processor 31 to the high computational processor 33 as part of the activation process.

Moreover, multiple parallel processors could be employed against the incoming data in the second stage, each of which multiple parallel processors could be programmed to detect one or more specific events. These multiple processors could be arranged serially as well to sequentially process the incoming data for one or more specific events.

By staging the digital analysis algorithms, the present invention achieves high sensitivity for alarm conditions with low computational throughput and low power consumption, while simultaneously achieving high specificity with more computationally intensive algorithms that only run occasionally, thereby achieving minimum power consumption with both high sensitivity and specificity.

Turning to FIG. 4, shown therein is an exemplary embodiment 40 of a method for monitoring a heart of a wearer of a body worn device, which outputs electrocardiogram signals or other heart related data signals.

In step 41, a first processing stage is employed to process real-time heart data to identify one or more potential alarm conditions, which first processing stage is optimized to minimize power consumption. The first processing stage could be a first operating mode (e.g., a low power consumption mode) of a processor, or the first processing stage could be a dedicated low power processor programmed to perform the first stage processing tasks.

In step 42, a second processing stage is employed to process data relating to the one or more potential alarm conditions to identify one or more actual alarm conditions among the one or more potential alarm conditions, which second processing stage is optimized to maximize throughput of the data. The second processing stage could be a second operating mode (e.g., a high throughput mode) of a processor, or the second processing stage could be a dedicated high throughput processor programmed to perform the second stage processing tasks.

In step 43, signal acquisition, a user interface, and alarm transmission tasks are managed with the first processing stage.

According to yet another aspect of the present invention, an exemplary embodiment of the detection algorithm will differentiate the alarm conditions to multiple levels of alarm alerts. Although there are many ways to differentiate the alarm conditions, the exemplary embodiment uses three alarm alert levels. So, in step 44, the one or more alarm conditions are differentiated among to multiple levels of alarm alerts with the second processing stage, which multiple levels of alarm alerts include a low level alert (e.g., indicates detection of one or more conditions that are related to technical aspects of a heart monitoring device), a medium level alert (e.g., indicates a medical condition has been detected in the patient that may not require immediate medical attention) and a high level alert (e.g., indicates a life threatening medical condition has been detected).

It should be noted that an alarm condition detected in the first stage that is subsequently determined in the second stage to be coincident with the presence of artifact reference signals can safely be ignored, since life threatening arrhythmias quickly result in an unconscious patient in whom artifact is unlikely. Similarly, normally elevated heart rates due to exertion will typically be accompanied by artifact signals, and will not reach alarm thresholds unless artifacts also contaminate the ECG signal. Thus, an alarm condition that is artifact-free and that is confirmed by the more advanced ECG analysis algorithms of stage two will result in performance of the multistage LTAD algorithm that is both sensitive and specific for the alarm conditions. Thus, the second stage algorithm can determine additional information about a potential alarm condition that can be used to filter out extraneous alarms.

In step 45, a technical call service center is alerted upon detecting a low level alert with the second processing stage. A low level alert implies notification of a technical call service center when the algorithm detects conditions that are related to technical aspects of the device, or that may be related to prolonged artifact conditions or other conditions implying impaired functioning of the device. A low level alert probably does not require medical attention. In a low level alert, the purpose of the call to the technical call center is to provide the user assistance in returning the device to a fully functioning state.

In step 46, a call service center (perhaps the same or different from the technical call service center) is alerted upon detecting a medium level alert with the second processing stage. A medium level alert implies notification of a call center to help assess the patient's medical status. A medium level alert may not require immediate medical attention, but the patient may be encouraged to call his physician, for example. Examples of a medium level alert are prolonged moderate tachycardia or bradycardia.

In step 47, a call center (perhaps the same or different from the call centers in steps 45 and 46) and/or emergency medical services is alerted upon detecting a high level alert with the second processing stage. A high level alert implies a life threatening arrhythmic condition that requires immediate medical attention. A high level alert may initiate a call to both a call center and emergency medical services. Examples of a high level alert are VF, extreme VT, or extreme bradycardia or asystole. These alerts can be accomplished via wireless communication (radio frequency transmission) or by notifying the patient to call a specific telephone number.

It should be noted that steps 43-47 of FIG. 4 could be added to the methods set forth above. For example, steps 43-47 could be added to the exemplary embodiment of the method of FIG. 2 after step 25.

Although various embodiments are specifically illustrated and described herein, it will be appreciated that modifications and variations of the invention are covered by the above teachings and are within the purview of the appended claims without departing from the spirit and intended scope of the invention. For example, two processing stages are discussed, however, three or more are also possible without departing from the scope of the present invention. Moreover, two or more processors may be employed—not just one as discussed in certain embodiments.

1. A heart monitoring apparatus comprising:
a memory storing a first set of programming instructions and a second set of programming instructions; and
a digital processor to be coupled to the memory and to receive real time data, said processor to be programmed by the first set of programming instructions to detect with optimum sensitivity one or more potential alarm conditions in the real time data, said processor when executing under the first set of programming instructions to be optimized to minimize power consumption, said processor when executing under the first set of programming instructions to activate the second set of programming instructions upon detection of one or more potential alarm conditions in the real time data, and said processor when executing under the second set of programming instructions to be optimized to maximize specificity for one or more alarm conditions.

2. The apparatus according to claim 1, wherein the processor comprises a variable clock speed processor, and a clock speed of the processor when executing under the first set of programming instructions is selected to minimize power consumption of the processor.

3. The apparatus according to claim 1, wherein the processor includes a variable clock speed, and a clock speed of the processor when executing under the second set of programming instructions is selected to maximize data throughput of the processor.

4. The apparatus according to claim 1, wherein the digital processor comprises:
a first digital processor to receive real time data, said first processor being programmed to detect with optimum sensitivity one or more potential alarm conditions in the real-time data, wherein said first processor is optimized to minimize power consumption; and
a second digital processor being programmed to maximize specificity for one or more alarm conditions, said second processor activated by the first processor upon detection of said one or more potential alarm conditions in the real time data.

5. The apparatus according to claim 4, wherein the first processor includes a clock speed selected to minimize power consumption.

6. The apparatus according to claim 4, wherein the second processor includes a clock speed selected to maximize data throughput.

7. A method for monitoring a heart comprising:
minimizing power consumption during a first stage of processing of the real-time data;
detecting one or more potential alarm conditions during the first stage of processing the real time data;
activating a second stage of processing of the real time data upon detecting said one or more potential alarm conditions; and
increasing data throughput during the second stage of processing to identify one or more alarm conditions among the one or more potential alarm conditions.

8. The method according to claim 7, further comprising:
maximizing specificity for one or more alarm conditions among the one or more potential alarm conditions during the second stage of processing of the real time data.

9. A method for monitoring a heart comprising:
sensing one or more potential alarm conditions with a first algorithm that is optimized to reduce power consumption; and
activating a second algorithm upon sensing one of said one or more potential alarm conditions to determine additional information regarding the sensed one of said one or more alarm conditions:

10. The method according to claim 9, wherein the additional information includes a presence of one or more artifacts.

11. The method according to claim 9, wherein the first algorithm detects one or more life-threatening arrhythmias among electrocardiogram signals, including one or more of the following: ventricular fibrillation, fast ventricular tachycardia, extreme bradycardia and asystole.

12. The method according to claim 11, wherein the first algorithm employs a QRS detector/counter for estimating heart rate, and one or more heart rate thresholds to identify the one or more life-threatening arrhythmias.

13. The method according to claim 9, wherein the second algorithm uses one or more independent estimates of the heart rate to confirm that one or more thresholds have been exceeded.

14. The method according to claim 9, wherein the second algorithm uses one or more parameters estimated from electrocardiogram signals related to ventricular fibrillation and fast ventricular tachycardia to identify an artifact among the electrocardiogram signals.

15. The method according to claim 9, wherein the second algorithm uses a signal derived from a common mode current to identify an artifact among the electrocardiogram signals

16. The method according to claim 9, wherein the second algorithm uses acceleration or patient impedance to identify an artifact among the electrocardiogram signals.

17. The method according to claim 9, further comprising differentiating (44) among the one or more alarm condition to multiple levels of alarm alerts with the second processor.

18. The method according to claim 17, wherein the multiple levels of alarm alerts include a low level alert, a medium level alert and a high level alert.

19. The method according to claim 18, wherein the low level alert indicates detection of one or more conditions that are related to technical aspects of a heart monitoring device.

20. The method according to claim 18, wherein the medium level alert indicates a medical condition has been detected in the patient that may not require immediate medical attention.

21. The method according to claim 18, further comprising alerting a call center upon detecting an alert.

22. A method for monitoring a heart comprising:
employing a first processing stage to process real-time heart data to identify one or more potential alarm conditions, wherein said first processor is optimized to minimize power consumption; and
employing a second processing stage to process data relating to the one or more potential alarm conditions to identify one or more actual alarm conditions among the one or more potential alarm conditions; wherein said second processor is optimized to maximize throughput of the data.

23. The method according to claim 22, further comprising managing signal acquisition, a user interface, and alarm transmission with the first processing stage.

24. The method according to claim 22, further comprising differentiating among the one or more alarm condition to multiple levels of alarm alerts with the second processing stage.

25. The method according to claim 22, wherein the first processing stage comprises a first digital processor executing a first programming and the second processing stage comprises a second digital processor executing a second programming.

26. The method according to claim 22, wherein the first processing stage comprises a digital processor executing a first programming and the second processing stage comprises a said digital processor executing a second programming.
